(19) United States
(12) Patent Application Publication
(10) Pub. No.: US 2017/0014589 A1
(43) Pub. Date: Jan. 19, 2017

(54) TRACHEAL INTUBATION DOUBLE TUBE

(71) Applicant: CATHOLIC UNIVERSITY INDUSTRY-ACADEMIC COOPERATION FOUNDATION, Seoul (KR)

(72) Inventor: Kyung Sool JANG, Incheon (KR)

(73) Assignee: CATHOLIC UNIVERSITY INDUSTRY-ACADEMIC COOPERATION FOUNDATION, Seoul (KR)

(21) Appl. No.: 15/120,551

(22) PCT Filed: Feb. 25, 2015
(86) PCT No.: PCT/KR2015/001843

§ 371 Date: Aug. 22, 2016

(30) Foreign Application Priority Data
Feb. 25, 2014 (KR) 10-2014-0021790

(51) Int. Cl. A61M 16/04; A61M 16/08

(52) U.S. Cl. CPC A61M 16/0427; A61M 16/0816; A61M 16/044; A61M 2205/0238; A61M 2205/0222

(57) ABSTRACT

The present invention relates to a tracheal intubation double tube that includes an outer tube that is inserted into a human body organ; and an inner tube that can be inserted and discharged by coming into close contact with an inner peripheral surface of the outer tube, and is fastened to and separated from the outer tube at a tip end portion.

### TECHNICAL FIELD

The present invention relates to a tracheal intubation double tube, and more particularly, to a tracheal intubation double tube that is capable of removing foreign matters in the tube by replacing only the inner tube, without inserting a new one into a critical patient.

### BACKGROUND ART

While treating all critical patients, a maintenance of smooth respiration is the most fundamental matter in the treatment of patients. Some critical patients require a maintenance of respiration through a tracheal intubation tube. In particular, unless patients suffering from pulmonary hemorrhage hemoptysis (lung hemorrhage), pulmonary edema and pneumonia remove blood and mucous sputum through a periodic suction, a problem in which coat of blood and mucous byproduct are stiffened to block the tracheal intubation tube may occur.

Furthermore, because substances which cause he blocking of such tracheal intubation tube also act as a cause of bacterial growth, they are known to contribute to the deterioration and the occurrence of pneumonia. In such a case, the patient shows several reactions due to the respiration disorder, and when there is a conscious, the patient is exposed to a very difficult situation.

At present, it is necessary to insert the new tracheal intubation tube into the patient again, and such a process may cause a great pain to the patient. In particular, such a process may act as great harm for a patient with clear consciousness and a patient suffering from a brain edema who rises in a venous pressure to be increased to a brain pressure due to a rise of an abdominal pressure. Therefore, there is a situation in which it is necessary to develop a tube capable of removing foreign matters in the tracheal intubation tube, without replacing a new one.

### DISCLOSURE

### Technical Problem

Thus, to solve the problems of conventional techniques as described above, an object of the present invention is to provide a tracheal intubation double tube that is capable of removing foreign matter in the tube by replacing only the inner tube, without inserting a new tracheal intubation tube into a critical patient.

### Technical Solution

According to an aspect of the present invention, there is provided a tracheal intubation double tube that includes an outer tube that is inserted into a human body organ; and an inner tube that can be inserted and discharged by coming into close contact with an inner peripheral surface of the outer tube, and is fastened to and separated from the outer tube at a tip end portion.

Fastening portions for each fastening may be provided at the tip end portions of the outer tube and the inner tube.

The fastening portions may include an outer fastening portion provided at the tip end portion of the outer tube and formed with a fastening groove; and an inner fastening portion provided with a fastening hook that is hooked on the fastening groove.

A respiratory connection port connected to a ventilator may be provided at the tip end portion of the inner tube.

A lubricating coating layer for insertion and discharging of the inner tube may be formed on the inner peripheral surface of the outer tube and the outer peripheral surface of the inner tube.

The lubricating coating layer may be formed by any one substance of polyurethane, vinyl resins, polyolefins, polyethylene, polypropylene, elastomers, polystyrene, polyesters, polyacrylates, polymethacrylates, silicone resins or polyamide polyamides.

The distal end portion of the inner tube may be formed to have a diameter that becomes narrower toward the distal end.

A balloon may be provided to be inflatable at the distal end portion of the outer tube.

### Advantageous Effects

According to an aspect of the present invention, because it is possible to remove foreign matters in the tube by replacing only the inner tube without inserting a new one into a critical patient, and only the inner tube may be periodically replaced while maintaining the outer tube as it is, it is possible to maintain a comfortable respiration in the patient, without pain.

### DESCRIPTION OF DRAWINGS

FIG. 1 is a perspective view of an outer tube according to the present invention.

FIG. 2 is a perspective view of the inner tube according to the present invention.

FIG. 3 is a perspective view illustrating the inner tube inserted into the outer tube in accordance with an embodiment of the present invention.

### BEST MODE

Hereinafter, embodiments of the invention will be described in detail with reference to the accompanying drawings. The drawings are attached hereto to help explain exemplary embodiments of the invention, and the present invention is not limited to the drawings and embodiments. In the drawings, some elements may be exaggerated, reduced in size, or omitted for clarity or conciseness.

Since the present invention may make various changes and may have several embodiments, specific embodiments will be illustrated in the accompanying drawings and will be described in detail in the detailed description.

However, this is not intended to limit specific embodiments of the present invention, and it should be understood to include all changes, equivalents and substitutes that are included within the spirit and scope of the present invention.

The terms used in this application are simply used for the purpose of describing particular embodiments and are not intended to limit the present invention. Singular representations include plural representations, unless they are expressed in a clearly different manner in the context. In this application, terms such as “including” or “having” should be understood to specify the presence of features, numbers, steps, operations, constituent elements, parts or the combination thereof, rather than excluding in advance the possibility of the presence or addition of one or more other features, numbers, steps, operations, constituent elements, parts or the combination thereof.

Hereinafter, an embodiment of the tracheal intubation double tube according to the present invention will be described in detail with reference to the accompanying drawings.

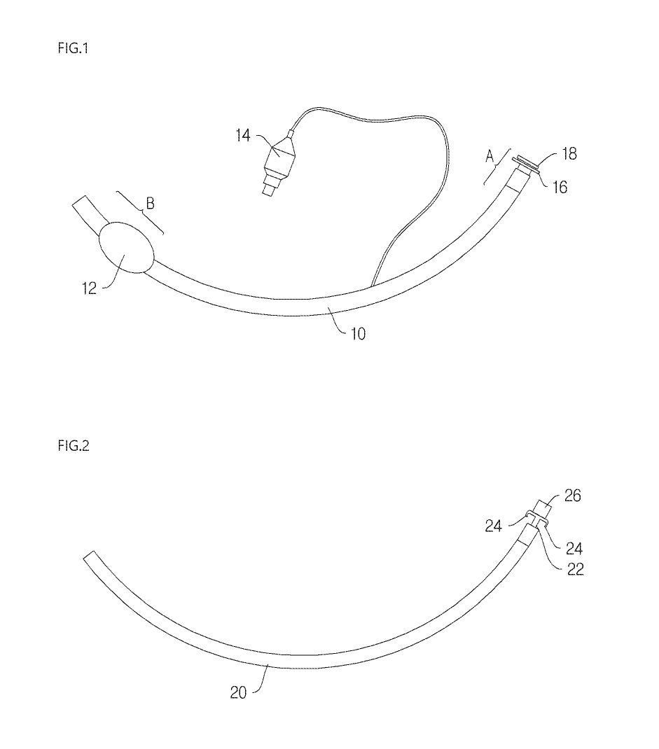

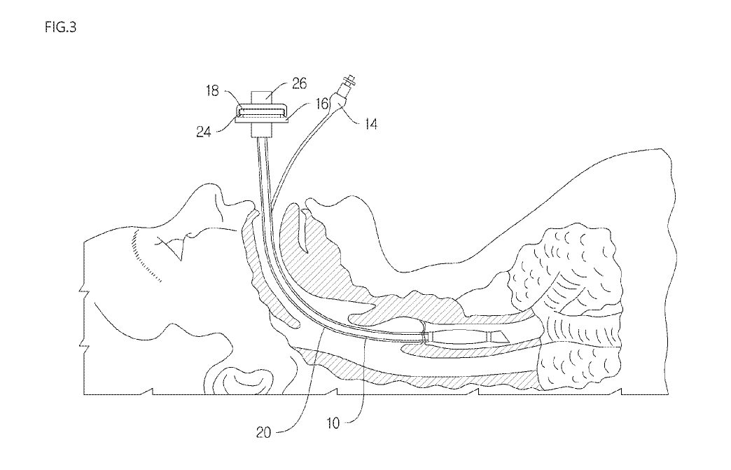

FIG. 1 is a perspective view of the outer tube according to the present invention, FIG. 2 is a perspective view of the inner tube according to the present invention, and FIG. 3 is a perspective view illustrating the inner tube inserted into the outer tube in accordance with an embodiment of the present invention.

As illustrated, the tracheal intubation double tube according to the present invention can include an outer tube 10 that is inserted into a human body organ; and an inner tube 20 that can be inserted and discharged by coming into close contact with an inner peripheral surface of the outer tube 10, and is fastened to and separated from the outer tube 10 at a tip end portion.

The outer tube 10 includes a tip end portion A which is exposed to the exterior of the human body in the inserted state, and a distal end portion B that is directly inserted into the human body organs. The outer tube 10 should be able to be smoothly inserted, without damaging the inner wall of the organ formed with bent portions and curved portions. Furthermore, the outer tube 10 needs to have predetermined rigidity so that folding does not occur in the bend portions and the curved portions of the organ in a state of being inserted to the desired location, and meanwhile, there is a need for flexibility that allows the outer tube to be inserted to the desired location, while not impairing inner wall of the organ.

The outer tube 10 may be formed in a variety of materials that are generally used in a medical tube, and preferably, may be formed of one or more selected from the group consisting of elastomer, silicone and soft resin. However, the outer tube may be formed of, not limited thereto, for example, polymerized material such as Nylon, Dacron, synthetic polyamide, expanded polytetrafluro-ethylene, polyethylene and ultra-high molecular weight fiber ultra-high molecular weight fibers of polyethylene, or stainless steel, cobalt-chromium alloy, titanium, titanium alloy or nickel-titanium shape memory alloys, or other materials.

Meanwhile, an inflatable balloon 12 is provided at the distal end portion B of the outer tube 10. The balloon 12 communicates with an air pump 14 provided outside to allow the inflow of air. The balloon 12 serves to fix the outer tube 10 in close contact with the inner wall of the organ, in inflating when the outer tube 10 is inserted to the desired location.

Next, referring to FIG. 2, the inner tube 20 comes in close contact with the inner circumferential surface, and can be coupled to the inside of the outer tube 10 so as to allow the insertion and discharge. The inner tube 20 has a slightly smaller diameter than the outer tube 10 so as to be inserted into the inner peripheral surface of the outer tube 10 while coming into a close contact therewith. The reason is in order to be able to easily remove the foreign matters in the tube, by allowing only the inner tube 20 to be replaced, without inserting a new one into a patient who has double tracheal intubation tubes in the present embodiment.

More specifically, in the state of inserting the tracheal intubation tube into the patient, the coat of blood and mucous byproduct are stiffened to block the inner tube 20. At this time, conventionally, although a surgery of discharging the entire tubes to the outside and inserting a new tracheal intubation tube has been performed, in this embodiment, since the blocking occurs only the inner tube 20, when replacing only the inner tube 20, the outer tube 10 can maintain the inserted state as it is.

Accordingly, it is possible to prevent a problem in which a patient suffers from a pain generated from the process of replacing a new tracheal intubation tube, and a secondary damage occurs. Furthermore, as described above, it is desirable to configure the inner tube 20 be in close contact with the inner peripheral surface of the outer tube 10. This is to prevent the coat of blood and mucous byproduct causing blockage of the organ from flowing between the outer tube 10 and inner tube 20.

Furthermore, the distal end portion of the inner tube 20 may be formed to have a diameter that becomes narrower toward the distal end in order to smoothly insert the inner tube 20 into the outer tube 10. When formed in this way, because the inner tube 20 to be replaced can more easily inserted into the outer tube 10, the outer tube 10 can be easily coupled to the inner tube 20.

Referring to FIGS. 1 and 2 again, fastening portions 16 and 22 for fastening each other are provided at the tip end portions A of the outer tube 10 and the inner tube 20. The fastening portions 16 and 22 may be made up of a separate injection-molded product and may be provided at each of the tip end portions of the outer tube 10 and the inner tube 20, but is not limited thereto, the fastening portions 16 and 22 may be formed integrally with the outer tube 10 and the inner tube 20.

The fastening portions 16 and 22 may include an outer fastening portion 16 which is provided at the tip end portion A of the outer tube 10 and is formed with a fastening groove 18; and an inner fastening portion 22 provided with a fastening hooks 24 fastened to a fastening groove 18. The fastening grooves 18 are formed to be recessed on both sides of the outer fastening portion 16, and each of a pair of fastening hooks 24 are fastened to the fastening grooves 18. Of course, in this embodiment, although the fastening groove 18 and the fastening hook 24 have been described as an example of the fastening portions 16 and 22, but is not limited thereto, and as long as it is possible to fasten the tip end portions of the outer tube 10 and the inner tube 20, any configuration may be adopted.

As explained above, while maintaining the state in which the outer tube 10 and the tip portion A of the inner tube 20 are fastened by the fastening portions 16 and 22, they can be inserted into the interior of the human body organ. Further, since the inner tube 20 is in a state of being fastened to the outer tube 10, the tubes can be inserted along the bent portions and the curved portions of the organ interior, while maintaining a predetermined stiffness. Of course, although it is not specifically illustrated in the drawings, a separate style may be inserted into the interior of the inner tube 20 together to guide the insertion of the tracheal intubation double pipe.

A respiratory connection portion 26 to which a ventilator (not shown) is connected may be provided at the tip end portion A of the inner tube 20. The respiratory connection portion 26 can be made in an approximately ring-shaped so that it can communicate with the inner tube 20. The respiratory connection portion 26 may be provided at the tip end portion A of the inner tube 20 as a separate injection-molded product, and may be formed integrally with the tip end portion A of the inner tube 20.

Meanwhile, a lubricating coating layer (not shown) for insertion and discharge of the inner tube 20 may be formed on the inner peripheral surface of the outer tube 10 or the outer peripheral surface of the inner tube 20. The lubricating coating layer is formed so that the inner tube 20 can be smoothly inserted and discharged, and substance having the hydrophilic properties may be coated on the inner peripheral surface of the outer tube 10 or the outer peripheral surface of or the inner tube 20. For example, on the outer peripheral surface of the inner tube 20, any one substance of polyurethane, vinyl resins, polyolefins, polyethylene, polypropylene, elastomers, polystyrene, polyesters, polyacrylates, polymethacrylates, silicone resins or polyamide polyamides may be coated. When the lubricating coating layer is formed on the inner peripheral surface of the outer tube 10 or the outer peripheral surface of the inner tube 20, the friction is reduced to easily insert the inner tube 20 into the outer tube 10 and to further facilitate the insertion by applying water using the hydrophilic properties to the surface.

Hereinafter, the operation process of the tracheal intubation double tube having the aforementioned configuration according to the present invention will be described in detail.

First, an operator can insert the double tube into the interior of the patient's organ in order to maintain a smooth respiration. At this time, the double tube is in a state in which the outer tube 10 and the inner tube 20 are fastened by the fastening portions 16 and 22, and a style (not shown) is inserted into the interior of the inner tube 20 to have a predetermined rigidity.

When the coat of blood and mucous byproduct are stiffened in the state in which the double tube is inserted, a problem that the interior of the inner tube 20 is blocked occurs. At this time, the operator separates the fastening portions 16 and 22 and discharges the inner tube 20 from the outer tube 10. The inner tube 20 can be more smoothly discharged by the lubricating coating layer.

In the state in which the inner tube 20 is completely discharged, the operator inserts a new inner tube 20 into the interior of the outer tube 10. In this way, since it is possible to replace only the new inner tube 20 in this embodiment, a patient does not surfer from the pain in the course of replacing the entire tube and can maintain the respiration in a stable state.

While the embodiments of the present invention have been described above in detail, the scope of the present invention is not limited thereto, and various modifications and improved aspects of those skilled in the art utilizing the basic concept of the present invention that are defined in the following claims also belong to the scope of the present invention.

1. A tracheal intubation double tube comprising:
an outer tube that is inserted into a human body organ; and
an inner tube that can be inserted and discharged by coming into close contact with an inner peripheral surface of the outer tube, and is fastened to and separated from the outer tube at a tip end portion.

2. The tracheal intubation double tube of claim 1, wherein fastening portions for each fastening are provided at the tip end portions of the outer tube and the inner tube.

3. The tracheal intubation double tube of claim 2, wherein the fastening portions comprises an outer fastening portion provided at the tip end portion of the outer tube and formed with a fastening groove; and
an inner fastening portion provided with a fastening hook that is hooked on the fastening groove.

4. The tracheal intubation double tube of claim 1, wherein a respiratory connection port connected to a ventilator is provided at the tip end portion of the inner tube.

5. The tracheal intubation double tube of claim 1, wherein a lubricating coating layer for insertion and discharging of the inner tube is formed on an inner peripheral surface of the outer tube and an outer peripheral surface of the inner tube.

6. The tracheal intubation double tube of claim 5, wherein the lubricating coating layer is formed by any one substance of polyurethane, vinyl resins, polyolefins, polyethylene, polypropylene, elastomers, polystyrene, polyesters, polyacrylates, polymethacrylates, silicone resins or polyamide polyamides.

7. The tracheal intubation double tube of claim 1, wherein a distal end portion of the inner tube is formed to have a diameter that becomes narrower toward the distal end.

8. The tracheal intubation double tube of claim 1, wherein a balloon is provided to be inflatable at the distal end portion of the outer tube.

9. The tracheal intubation double tube of claim 2, wherein a balloon is provided to be inflatable at the distal end portion of the outer tube.

10. The tracheal intubation double tube of claim 3, wherein a balloon is provided to be inflatable at the distal end portion of the outer tube.

11. The tracheal intubation double tube of claim 4, wherein a balloon is provided to be inflatable at the distal end portion of the outer tube.

12. The tracheal intubation double tube of claim 5, wherein a balloon is provided to be inflatable at the distal end portion of the outer tube.

13. The tracheal intubation double tube of claim 6, wherein a balloon is provided to be inflatable at the distal end portion of the outer tube.

14. The tracheal intubation double tube of claim 7, wherein a balloon is provided to be inflatable at the distal end portion of the outer tube.
